(19) United States
(12) Patent Application Publication
(10) Pub. No.: US 2005/0090009 A1
(43) Pub. Date: Apr. 28, 2005

(54) Compositions of stabilized DNA for coating microprojctions

(76) Inventors: Michel J.N. Cormier, Mountain View, CA (US); Georg Widera, Palo Alto, CA (US); Mahmoud Ameri, Fremont, CA (US)

Correspondence Address: Francis Law Group, 1942 Embarcadero, Oakland, CA, 94606, US

(21) Appl. No.: 10/972,230

(22) Filed: Oct. 21, 2004

Related U.S. Application Data
(60) Provisional application No. 60/514,533, filed on Oct. 23, 2003.

(51) Int. Cl. A61K 48/00; C12N 15/87

(52) U.S. Cl. 435/459; 514/44

(57) ABSTRACT

The present invention provides methods and compositions for stabilizing dried nucleic acids with carbohydrates such as non-reducing sugars, polysaccharides, and reducing sugars. Preferably, the stabilized nucleic acids are coated on a microprojection member for transdermal delivery. The invention further provides compositions and methods that involve the use of DNase inhibitors to stabilize dried nucleic acids delivered directly into bodily tissues.

### CROSS-REFERENCE TO RELATED APPLICATIONS

This application claims the benefit of U.S Provisional Application No. 60/514,533, filed Oct. 23, 2003.

### FIELD OF THE PRESENT INVENTION

The present invention relates to methods and compositions for retarding the degradation of nucleic acids. More particularly, the invention relates to methods and compositions for coating microprojections to transdermally deliver such nucleic acids.

### BACKGROUND OF THE INVENTION

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

As an alternative, transdermal delivery provides for a method of administering biologically active agents that would otherwise need to be delivered via hypodermic injection, intravenous infusion or orally. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the biologically active agent during transdermal administration. Indeed, many drugs such as aspirin have an adverse effect on the digestive tract.

The word “transdermal” is used herein as a generic term referring to passage of an agent across the skin layers. The word “transdermal” refers to delivery of an agent (e.g., a nucleic acid or other therapeutic agent such as a drug) through the skin to the local tissue or systemic circulatory system without substantial cutting or piercing of the skin, such as cutting with a surgical knife or piercing the skin with a hypodermic needle. The outermost skin layer is formed from flat, dead cells filled with keratin fibers (keratinocytes) surrounded by lipid bilayers. The highly-ordered structure of the lipid bilayers confers a relatively impermeable character to the stratum corneum. Thus, one of the most significant challenges in a transdermal delivery system is transporting the agent through this portion of the skin.

Transdermal agent delivery includes delivery via passive diffusion as well as by external energy sources including electricity (e.g., iontophoresis) and ultrasound (e.g., phonophoresis). While agents do diffuse across both the stratum corneum and the epidermis, the rate of diffusion through the stratum corneum is often the limiting step for the reasons discussed above. Many compounds, in order to achieve a therapeutic dose, require higher delivery rates than can be achieved by simple passive transdermal diffusion.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal agent flux has been limited, particularly for larger molecules.

Active transport systems use an external energy source to assist agent flux through the stratum corneum. One such enhancement for transdermal agent delivery is referred to as “electrotransport.” This mechanism uses an electrical potential, which results in the application of electric current to aid in the transport of the agent through a body surface, such as skin. Other active transport systems use ultrasound (phonophoresis) and heat as the external energy source.

There also have been many techniques and systems developed to mechanically penetrate or disrupt the outermost skin layers thereby creating pathways into the skin in order to enhance the amount of agent being transdermally delivered. Illustrative are skin scarification devices, or scarifiers, which typically provide a plurality of tines or needles that are applied to the skin to scratch or make small cuts in the area of application. The agent, such as a vaccine, is applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

Scarifiers have been suggested for intradermal vaccine delivery, in part, because only very small amounts of the vaccine need to be delivered into the skin to be effective in immunizing the patient. Further, the amount of vaccine delivered is not particularly critical since an excess amount also achieves satisfactory immunization.

A major drawback associated with the use of a scarifier to deliver an active agent is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Other devices which use tiny skin piercing elements to enhance transdermal agent delivery are disclosed in European Patent EP 0407063A1, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., U.S. Pat. No. Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety. These devices use piercing elements of various shapes and sizes to pierce the outermost layer (i.e., the stratum comeum) of the skin. The piercing elements disclosed in these references generally extend perpendicularly from a thin, flat member, such as a pad or sheet.

The piercing elements in some of these devices are extremely small, some having dimensions (i.e., a microblade length and width) of only about 25-400 μm and a microblade thickness of only about 5-50 μm. These tiny piercing/cutting elements make correspondingly small microslits/microcuts in the stratum corneum for enhanced transdermal agent delivery therethrough.

Generally, the disclosed systems include a reservoir for holding the drug and also a delivery system to transfer the drug from the reservoir through the stratum comeum, such as by hollow tines of the device itself. Alternatively, a formulation containing the active agent can be coated on the microprojections themselves. Such an approach has been disclosed in published U.S. patent applications No. 2002/0132054, 2002/0193729, 2002/0177839, 2002/0128599, and Ser. No. 10/045,842, which are fully incorporated by reference herein. This approach eliminates the necessity of a separate physical reservoir and developing an agent formulation or composition specifically for the reservoir.

Thus, using a microprojection device to transdermally deliver an agent coated on the microprojections confers a number of benefits. Accordingly, it is an object of this invention to provide methods and compositions for facilitating transdermal delivery of biologically active agents.

It is a further object of the invention to provide a coating for improved transdermal delivery of nucleic acids.

It is another object of the invention to provide a nucleic acid formulation that exhibits reduced degradation.

Similarly, it is an object of the invention to retard degradation of a nucleic acid by preparing stabilized formulations for solid coatings.

### SUMMARY OF THE INVENTION

In accordance with the above objects and those that will be mentioned and will become apparent below, one aspect of the invention is directed to a solid coating comprising a dried formulation of a stabilizing agent and nucleic acid applied to a solid substrate wherein the stabilizing agent retards degradation of the dried nucleic acid. Preferably, the solid substrate comprises a microprojection member. Also preferably, the solid coating has a thickness in the range of approximately 1 to 50 micrometers.

In certain embodiments of the invention, the stabilizing agent is selected from the group consisting of a non-reducing sugar, a polysaccharide, and a reducing sugar. In embodiments comprising a non-reducing sugar, the stabilizing agent is preferably selected from the group consisting of sucrose, trehalose, stachyose, and raffinose. In embodiments comprising a polysaccharide, the stabilizing agent is preferably selected from the group consisting of dextran, soluble starch, dextrin, and inulin. In embodiments comprising a reducing sugar, the stabilizing agent is preferably selected from the group consisting of apiose, arabinose, lyxose, ribose, xylose, digitoxose, fucose, quercitol, quinovose, rhamnose, allose, altrose, fructose, galactose, glucose, gulose, hamamelose, idose, mannose, tagatose, primeverose, vicianose, rutinose, scillabiose, cellobiose, gentiobiose, lactose, lactulose, maltose, melibiose, sophorose, and turanose.

In another aspect of the invention, the nucleic acid is selected from the group consisting of double-stranded DNA, single-stranded DNA, and RNA. In one embodiment, a preferred nucleic acid is plasmid DNA.

Presently preferred embodiments solid coatings of the invention include the stabilizing agent comprising in the range of approximately of 10% to 80% by total dry weight, and more preferably in the range of approximately 20% to 80%.

Additionally, the solid coatings of the invention can further comprise one or more surface active agents up to 10% by total dry weight. Preferably, the surface active agents are selected from the group consisting of Polysorbate 20, Polysorbate 80, and sodium dodecyl sulfate.

Alternatively, or in combination with surface active agents, the formulation can further comprise a buffering agent in the range of approximately 0.5% to 20% by total dry weight. Such buffering agents are preferably selected from the group consisting of phosphoric acid, citric acid, and TRIS.

In a preferred embodiment of the invention, the formulation has a nucleic acid in the range of approximately 20% to 80% by total dry weight and a stabilizing agent in the range of approximately 10% to 80% by total dry weight.

In another embodiment ofthe invention, the nucleic acid is DNA and the formulation further comprises a DNase inhibitor, wherein said DNase inhibitor retards degradation of the DNA following delivery of said solid coating into or through skin using said microprojection member. Preferably, the DNase inhibitor is selected from the group consisting of aurintricarboxylic acid, EDTA, EGTA, propamidine, and DMI-2. Also preferably, the DNase inhibitor is in the range of approximately 1% to 20% by total dry weight of the formulation.

In a presently preferred embodiment, the formulation comprises the nucleic acid in the range of approximately 20% to 80% by total dry weight, the stabilizing agent in the range of approximately 10% to 80% by total dry weight and the DNase inhibitor in the range of approximately 1% to 20% by total dry weight.

In each of the noted embodiments, the formulation can also comprise a surface active agent and/or a buffering agent as described above.

In another embodiment, the coating formulations include a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In a further embodiment of the invention, the coating formulation includes at least one antioxidant, which can be sequestering, such as sodium citrate, citric acid, EDTA (ethylene-dinitrilo-tetraacetic acid) or free radical scavengers, such as ascorbic acid, methionine, sodium ascorbate, and the like. Presently preferred antioxidants include EDTA and methionine.

The invention is also directed to methods of stabilizing nucleic acids. In certain embodiments, the method comprises mixing a formulation of nucleic acid with a stabilizing agent and dry-coating the formulation onto a solid substrate, wherein the stabilizing agent retards degradation of the nucleic acid. Preferably, the solid substrate is a microprojection member.

In a further embodiment of the invention, the method comprises applying the coated microprojection member to a subject to transdermally deliver the nucleic acid.

Preferably, the methods of the invention comprise mixing a stabilizing agent selected from the group consisting of a non-reducing sugar, a polysaccharide, and a reducing sugar with the nucleic acid.

Also preferably, the methods of the invention comprise mixing a nucleic acid selected from the group consisting of double-stranded DNA, single-stranded DNA, and RNA.

In additional embodiments, the formulation can further comprise up to 10% by total dry weight of one or more surface active agents selected from the group consisting of polysorbate 20, polysorbate 80, and sodium dodecyl sulfate and/or a buffering agent in the range of approximately 0.5% to 20% by total dry weight, wherein the buffering agent is selected from the group consisting of phosphoric acid, citric acid, and TRIS.

In embodiments wherein the nucleic acid is DNA, the formulation can further comprise a DNase inhibitor as discussed above, preferably selected from the group consisting of aurintricarboxylic acid, EDTA, EGTA, propamidine, and DMI-2. Such formulations can also comprise a surface acting agent and/or a buffering agent as discussed above.

In the noted embodiments, the methods of the invention preferably comprise dry-coating the formulations on a microprojection member. Additionally, such methods further comprise applying the microprojection member to a subject to transdermally deliver the nucleic acid, wherein the DNase inhibitor retards degradation of the nucleic acid following delivery.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 depicts the percentage of supercoiled plasmid DNA in solution or dry-coated onto glass or a titanium microprojection array (S250) after storage at 4° C. or −20° C. for one to four weeks;

FIG. 2 depicts the percentage of supercoiled plasmid DNA dry-coated onto a titanium microprojection array (S250) after storage at 4° C. for one to eight weeks. The formulations used for coating contained no sucrose (CONTROL) or sucrose at the indicated concentrations (wt. %);

FIG. 3 is a perspective view of a microprojection member, according to the invention; and

FIG. 4 is a perspective view of the microprojection member shown in FIG. 3 having a coating deposited on the microprojections, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a”, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be formulated in the coatings, resulting in co-delivery of the biologically active agents.

The term “biologically active agent”, as used herein, refers to a composition of matter or mixture containing nucleic acids, such as oligonucleotides and polynucleotides.

The term “nucleic acid”, as used herein, includes double-stranded DNA, single-stranded DNA, and RNA, and plasmid DNA.

It is to be understood that more than one biologically active agent can be incorporated into the agent source and/or coatings of this invention, and that the use of the term “active agent” in no way excludes the use of two or more such active agents.

As used herein, the term “stabilizing agent” refers to any substance that retards or slows, to any measurable degree, the degradation of nucleic acid.

As used herein, the term “dried”, as it relates to nucleic acid, refers to nucleic acid that is substantially free of liquid.

As used herein, the terms “retards”, “retard”, and all variations thereof, refer to slowing by any measurable degree the degradation of nucleic acid.

As used herein, the term “degradation”, as it relates to nucleic acid, refers to any change in the structure of the nucleic acid by which the integrity of the intact structure is compromised, such as, for example, loss of supercoiled structure.

As used herein, the term “microprojection array,” “microprojection member,” “micro needle array device,” and the like, all refer to a device for delivering an active agent into or through the skin that comprises a plurality of microprojections on which the active agent can be dry-coated. The term “microprojections” refers to piercing elements that are adapted to pierce or cut through the stratum corneum into the underlying epidermis layer, or epidermis and dermis layers, of the skin of a living animal, particularly a human. The piercing elements should not pierce the skin to a depth that causes bleeding. Typically the piercing elements have a blade length of less than 1000 μm, and preferably less than 500 μm. The microprojections typically have a width of about 75 μm to 500 μm and a thickness of about 5 μm to 50 μm.

The microprojections can be formed in different shapes, such as needles, hollow needles, blades, pins, punches, and combinations thereof. Micro needle array devices are described, for example, in U.S. patent application Publication No. 2002/0132054, incorporated herein by reference in its entirety.

As used herein, the terms “mixing,” “mix,” “adding,” and “add,” and all variations thereof, refer to any means that directly or indirectly cause placement together of moieties or components, such that the moieties or components come into close proximity to each other. The terms include acts such as placing the moieties or components together in a container, combining the moieties or components, contacting the moieties or components, or stirring, vortexing, or agitating the moieties or components together. The term “mixture” refers to moieties or components that have been placed together in close proximity.

As used herein, the term “dry-coating” refers to any process by which a solution that contains one or more agents of interest is applied to a surface of a solid substrate and by which substantially all of the liquid is then removed from the solution of the one or more agents of interest. The terms “dry-coated” and “dry-coat,” and all variations thereof refer to the resultant solid coating produced by the dry coating process.

As used herein, the term “solid”, as it relates to coatings, refers to coatings that are essentially non-porous or non-particulate in nature. Solid coatings do not include compositions that have been lyophilized, which are porous in nature. Solid coatings also do not include particles formed from spray drying processes, which are particulate in nature.

As used herein, the term “solid substrate” refers to and includes any material upon which nucleic acid can be dry-coated, such as the microprojections of a microprojection member.

As used herein, the terms “deliver,” “delivering,” and all variations thereof, refer to and include any means by which an active agent can be administered into or through the skin.

As used herein, the term “thickness,” as it relates to solid coatings, refers to the average thickness of a solid coating as measured over substantially all of the portion of a solid substrate that is covered with the solid coating.

The term “microprojections”, as used herein, refers to piercing elements which are adapted to pierce or cut through the stratum comeum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

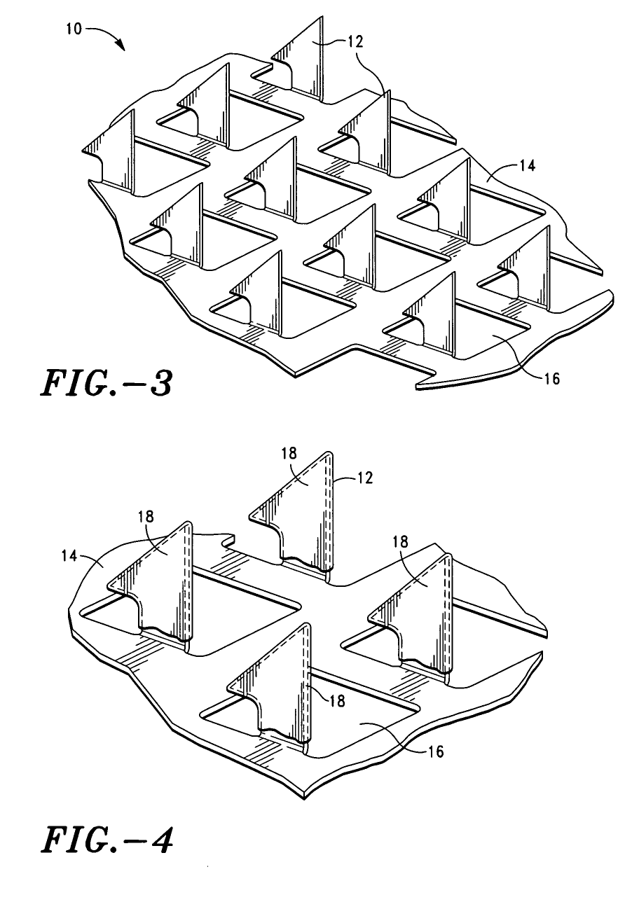

The term “microprojection member”, as used herein, generally connotes a microprojection array comprising a plurality of microprojections arranged in an array for piercing the stratum comeum. The microprojection member can be formed by etching or punching a plurality of microprojections from a thin sheet and folding or bending the microprojections out of the plane of the sheet to form a configuration, such as that shown in FIG. 3. The microprojection member can also be formed in other known manners, such as by forming one or more strips having microprojections along an edge of each of the strip(s) as disclosed in U.S. Pat. No. 6,050,988, which is hereby incorporated by reference in its entirety.

The present invention relates to the surprising discovery that dried nucleic acids can be stabilized with carbohydrates, such as non-reducing sugars, polysaccharides, and reducing sugars. Applicants have advantageously found that the degradation of dried nucleic acids can be prevented or measurably slowed by formulating the nucleic acids with one or more carbohydrates.

Nucleic acids are dried for many varied reasons, and dried nucleic acids are used in various applications and methodologies. Nucleic acids are sometimes dried prior to storage. Nucleic acids can also be dry-coated onto solid substrates, including delivery devices such as microprojection members, prior to administration to a patient or test subject. Applicants have developed methods and formulations for stabilizing nucleic acids in dried form based upon the surprising discovery that carbohydrates stabilize dried nucleic acids. Applicants' methods and compositions can be used to prevent or retard the degradation of nucleic acids that are dry-coated onto solid substrates, including delivery devices such as microprojection members. Applicants' methods and formulations enable dry-coated nucleic acids to be stably stored at 4° C. or room temperature for extended periods of time such as, for example, weeks to years.

Applicants have also advantageously discovered that dry-coated nucleic acids delivered directly into or through the skin can be further stabilized by formulating the nucleic acids with one or more DNase inhibitors. Naked DNA delivered directly into or through the skin is rapidly degraded by DNases present in the interstitial space of the skin. DNase inhibitors prevent or measurably retard the degradation of nucleic acids. Applicants have developed methods and compositions for stabilizing dried nucleic acids delivered directly into or through the skin or other tissues that involve formulating the dried nucleic acids with one or more DNase inhibitors, which increases the likelihood of cellular uptake of the nucleic acid and expression of one or more exogenous genes of interest.

Certain embodiments of the present invention relate to methods for retarding the degradation of dried nucleic acids that comprise mixing the nucleic acid with one or more stabilizing agents and dry coating the mixture onto a solid substrate. Other embodiments of the invention relate to compositions, including solid coatings, which comprise one or more stabilizing agents and nucleic acid. Nucleic acid stabilizing agents suitable for use in the methods and compositions of the invention include carbohydrates such as, for example, non-reducing sugars, polysaccharides, and reducing sugars. Other non-carbohydrate nucleic acid stabilizing agents are familiar to those of skill in the art and can be used, in certain embodiments of the compositions and methods of the invention, in combination with one or more carbohydrate stabilizing agents.

Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and inulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like. Particularly preferred stabilizing agents include, for example, sucrose.

In certain embodiments of the invention, the stabilizing agent is in the range of approximately 10% to 80% by total dry weight of the compositions and solid coatings. In certain more preferred embodiments, the stabilizing agent is in the range of approximately 20% to about 75% by total dry weight of the compositions and solid coatings. In even more preferred embodiments, the stabilizing agent is in the range of approximately 30% to about 70% by total dry weight of the compositions and solid coatings.

Nucleic acids that can be stabilized in dried form according to the methods of the invention, and nucleic acids that the compositions and dry coatings of the invention comprise, include single-stranded and double-stranded nucleic acids, such as, for example, single and double-stranded DNA and RNA, including, for example, supercoiled plasmid DNA; linear plasmid DNA; cosmids; bacterial artificial chromosomes (BACs); yeast artificial chromosomes (YACs); mammalian artificial chromosomes; DNA oligonucleotides, such as, for example, CpG containing oligonucleotides; DNAzymes; mRNA, antisense oligonucleotides, ribozymes, and siRNA (RNAi). The size of the nucleic acid can range from less than about 10 nucleotides to thousands of kilobases. In addition, in certain embodiments of the invention, the nucleic acid can be coupled with a proteinaceous agent or can include one or more chemical modifications, such as, for example, phosphorothioate moieties.

The nucleic acids can be used, for example, as adjuvants (immuno-stimulatory sequences), as immuno-suppressing agents, or as immuno-modulatory agents and can be co-formulated with protein or DNA vaccines, or administered separately either before or after protein or DNA immunization. The nucleic acids thus find application, for example, in the fields of infectious diseases, cancers, allergies, and inflammatory diseases.

As will be appreciated by one having ordinary skill in the art, with few exceptions, alum-adjuvanted vaccine formulations typically lose potency upon freezing and drying. To preserve the potency and/or immunogenicity of the alum-adsorbed vaccine formulations of the invention, the noted formulations can be further processed as disclosed in Provisional Application No. ______ [Attorney Docket No. ALZ 5156 PSP1, filed Sep. 28, 2004]; which is expressly incorporated by reference herein in its entirety.

In certain embodiments of the invention, the nucleic acid is in the range of approximately 20% to 80% by total dry weight of the compositions and solid coatings. In certain more preferred embodiments, the nucleic acid is in the range of approximately 25% to 75% by total dry weight of the compositions and solid coatings. In even more preferred embodiments, the nucleic acid is in the range of approximately 30% to 70% by total dry weight of the compositions and solid coatings.

Certain embodiments of the present invention relate to methods for retarding the degradation of dried nucleic acids that comprise mixing the nucleic acid with one or more stabilizing agents and one or more surface active agents and dry coating the mixture onto a solid substrate. Other embodiments of the invention relate to compositions, including solid coatings, that comprise one or more stabilizing agents, nucleic acid, and one or more surface active agents. Surface active agents suitable for use in the methods and compositions of the invention include, for example, negatively charged surfactants such as sodium dodecylsulfate; positively charged surfactants such as cetylpyridinium chloride, TMAC, and benzalkonium chloride; and neutral surfactants such as polysorbate, sorbitan, and laureth. Preferred surface active agents include, for example, polysorbate 20, polysorbate 80, and sodium dodecyl sulfate.

In certain embodiments of the invention, one or more surface active agents comprise up to approximately 10% by total dry weight of the compositions and solid coatings. In certain more preferred embodiments of the invention, one or more surface active agents are in the range of approximately 0.01% to 10% by total dry weight of the compositions and solid coatings of the invention. In even more preferred embodiments, one or more surface active agents are in the range of approximately 0.03% to 3% by total dry weight of the compositions and solid coatings of the invention.

In other embodiments, the invention relates to methods for retarding the degradation of dried nucleic acids that comprise mixing the nucleic acid with one or more stabilizing agents, one or more buffering agents, and/or one or more surface active agents and dry-coating the mixture onto a solid substrate. In certain other embodiments, the invention relates to compositions, including solid coatings, that comprise one or more stabilizing agents, nucleic acid, one or more buffering agents and/or one or more surface active agents. Buffering agents suitable for use in the methods and compositions of the invention include, for example, acetic acid, propionic acid, pentanoic acid, citric acid, succinic acid, glycolic acid, gluconic acid, glucuronic acid, lactic acid, malic acid, pyruvic acid, tartaric acid, tartronic acid, fumaric acid, glutamic acid aspartic acid, ammonia, morpholine, imidazole, monoethanolomine, diethanolamine, triethanolamine, tromethamine, methylglucamine, glucosamine, MES, ADA, PIPES, ACES, MOPS, TES, HEPES, HEPPS, histidine, and other buffering agents having an isoelectric pH at which the buffering agent contains no net charge (pI) of between 3 and 11.

Buffering agents suitable for use in the methods and compositions of the invention also include dipeptide buffers having a pI of between 5 and 9 as described, for example, in European Patent Application Publication Number EP 1 028 706 B1, U.S. patent application Publication No. 2002/0058608, and PCT Patent Application Publication Number WO 99/24015, incorporated herein by reference in their entireties. Examples of suitable dipeptide buffering agents include Gly-His and His-Gly. Particularly preferred buffering agents include, for example, phosphoric acid, citric acid, and TRIS.

In certain embodiments of the invention, one or more buffering agents are in the range of approximately 0.1% to 30% by total dry weight of the compositions and solid coatings. In certain more preferred embodiments, one or more buffering agents are in the range of approximately 0.5% to 20% by total dry weight of the compositions and solid coatings. In even more preferred embodiments, one or more buffering agents are in the range of approximately 1% to 10% by total dry weight of the compositions and solid coatings.

Certain other embodiments of the invention relate to methods for retarding the degradation of dried DNA that comprise mixing the DNA with one or more stabilizing agents, one or more DNase inhibitors, and optionally with one or more surface active agents and/or buffering agents, and dry coating the mixture onto a solid substrate, such as, for example, a micro needle array device. In certain embodiments of the invention, the methods further comprise delivering the dry-coated mixture into or through the skin using, for example, a micro needle array device, or needle.

Other embodiments of the invention relate to compositions, including solid coatings, comprising DNA, one or more stabilizing agents, one or more DNase inhibitors, and optionally one or more surface active agents and/or buffering agents. DNase inhibitors that can be used in the compositions and methods of the invention include, for example, both extracellular and intracellular DNase inhibitors. Preferred extracellular DNase inhibitors include, for example, aurintricarboxylic acid (ATA); EDTA; EGTA; and propamidine, which is an aromatic bisamidinium compound that selectively binds to the minor groove of DNA at AT stretches. Preferred intracellular DNase inhibitors include, for example, DMI-2, which is a polyketice metabolite of Streptomyces sp. Strain 560.

In certain embodiments of the invention, the DNase inhibitor is in the range of approximately 0.1% to 30% by total dry weight of the compositions and solid coatings of the invention. In certain more preferred embodiments, the DNase inhibitor is in the range of approximately 1% to 20% by total dry weight of the compositions and solid coatings. In even more preferred embodiments, the DNase inhibitor is in the range of approximately 2% to 10% by total dry weight of the compositions and solid coatings.

In certain embodiments, the invention relates to methods for retarding the degradation of nucleic acid that comprise mixing the nucleic acid with one or more stabilizing agents and dry coating the mixture onto a solid substrate. Other embodiments of the invention relate to compositions, including solid coatings, which comprise one or more stabilizing agents and nucleic acid. In preferred embodiments, the compositions take the form of a solid coating comprising one or more stabilizing agents and nucleic acid. In certain embodiments, nucleic acid and one or more stabilizing agents, optionally in combination with one or more surface active agents, one or more buffering agents, and/or one or more DNase inhibitors, are dry-coated onto a surface of a solid substrate. Solid substrates on which the compositions can be dry-coated include, for example, the microprojections of microprojection members; needles of any kind; microtiter plates; test tubes of any size, shape, or composition; and any medium on or in which nucleic acids can be stored. In preferred embodiments of the invention, the compositions are dry-coated onto the microprojections of a microprojection member.

Microprojection members have a plurality of skin piercing microprojections that can be dry-coated with an active agent, such as nucleic acid. When the microprojections penetrate the skin, the nucleic acid is delivered into the interstitial space and is solubilized. Microprojection members, and processes for coating such devices with an active agent, are described, for example, in U.S. patent application Publication No. 200/0132054 and PCT Application Publication Number WO 02/074173, incorporated herein by reference in their entireties.

The desired thickness of the solid coating on the microprojections of a micro needle array device is dependent upon the density of the microprojections per unit area and the viscosity and concentration of the coating composition as well as the coating method chosen. Coating thickness is referred to as an average coating thickness measured over the portion of the microprojection that has been coated. In general, coating thickness is preferably less than 50 μm since thicker coatings have a tendency to slough off the microprojections upon stratum comeum piercing. In certain embodiments of the invention, the dry, solid coatings have a thickness in the range of approximately 1 to 50 micrometers, preferably in the range of approximately 5 to 30 micrometers.

Referring now to FIG. 3, there is shown one embodiment of stratum comeum-piercing microprojection member 10 for use with the present invention. As illustrated in FIG. 3, the member 10 includes a plurality of microprojections 12 that are adapted to pierce into and through the stratum comeum.

The microprojections 12 typically extend at substantially a 90° angle from a sheet 14 having openings 16. The microprojections 12 are preferably formed by etching or punching a plurality of microprojections 12 from a thin metal sheet 14 and bending the microprojections 12 out of a plane of the sheet.

Referring now to FIG. 4, there is shown the microprojection member 10 having a solid coating 18 disposed on the microprojections 12. According to the invention, the coating 18 can partially or completely cover the microprojections 12. The coating 18 can also be applied before or after the microprojections 12 are formed.

Other microprojection members that can be used with the present invention are formed by etching silicon using silicon chip etching techniques or by molding plastic using etched micro-molds. Silicon and plastic microprojection members are disclosed in Godshall et al., U.S. Pat. No. 5,879,326; the disclosure of which is incorporated by reference herein.

According to the invention, the coating 18 can be applied to the microprojections 12 by a variety of known methods. Preferably, the coating is only applied to those portions the microprojection member 10 or microprojections 12 that pierce the skin (e.g., tips).

One such coating method comprises dip-coating. Dip-coating can be described as a means to coat the microprojections by partially or totally immersing the microprojections 12 into a coating solution. By use of a partial immersion technique, it is possible to limit the coating 18 to only the tips of the microprojections 12.

A further coating method comprises roller coating, which employs a roller coating mechanism that similarly limits the coating 18 to the tips of the microprojections 12. The roller coating method is disclosed in U.S. application Ser. No. 10/099,604 (Pub. No. 2002/0132054), which is incorporated by reference herein in its entirety.

As discussed in detail in the noted application, the disclosed roller coating method provides a smooth coating that is not easily dislodged from the microprojections 12 during skin piercing.

According to the invention, the microprojections 12 can further include means adapted to receive and/or enhance the volume of the coating 18, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 12. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

As indicated, according to one embodiment of the invention, the coating formulations applied to the microprojection member 10 to form dry coatings can comprise aqueous and non-aqueous formulations having at least one biologically active agent. According to the invention, the biologically active agent can be dissolved within a biocompatible carrier or suspended within the carrier.

According to the invention, the coating formulations preferably include at least one wetting agent. As is well known in the art, wetting agents can generally be described as amphiphilic molecules. When a solution containing the wetting agent is applied to a hydrophobic substrate, the hydrophobic groups of the molecule bind to the hydrophobic substrate, while the hydrophilic portion of the molecule stays in contact with water. As a result, the hydrophobic surface of the substrate is coated with hydrophobic groups of the wetting agent, making it susceptible to wetting by the solvent. Wetting agents include surfactants as well as polymers presenting amphiphillic properties.

In one embodiment of the invention, the coating formulations include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4. Most preferred surfactants include Tween 20, Tween 80, and SDS.

Preferably, the concentration of the surfactant is up to 10% by total dry weight of the solid coating. More preferably in the range of approximately 0.001-2 wt. % of the solid coating formulation.

The coatings of the invention can further include a vasoconstrictor, such as those disclosed in Co-Pending U.S. application Ser. Nos. 10/674,626 and 60/514,433, which are incorporated by reference herein in their entirety. As set forth in the noted Co-Pending Applications, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

According to the invention, the coating formulations can also include a non-aqueous solvent, such as ethanol, chloroform, ether, propylene glycol, polyethylene glycol and the like, dyes, pigments, inert fillers, permeation enhancers, excipients, and other conventional components of pharmaceutical products or transdermal devices known in the art.

Other known formulation adjuvants can also be added to the coating formulations as long as they do not adversely affect the necessary solubility and viscosity characteristics of the coating formulation and the physical integrity of the dried coating.

Preferably, the coating formulations have a viscosity less than approximately 500 centipoise and greater than 3 centipoise in order to effectively coat each microprojection 12. More preferably, the coating formulations have a viscosity in the range of approximately 3-200 centipoise.

According to the invention, the desired coating thickness is dependent upon the density of the microprojections per unit area of the sheet and the viscosity and concentration of the coating composition as well as the coating method chosen. Preferably, the coating thickness is less than 50 microns.

As stated, in certain embodiments of the invention, the dry, solid coatings have a thickness in the range of approximately 1 to 50 micrometers, preferably in the range of approximately 5 to 30 micrometers.

In all cases, after a coating has been applied, the coating formulation is dried onto the microprojections 12 by various means. In a preferred embodiment of the invention, the coated microprojection member 10 is dried in ambient room conditions. However, various temperatures and humidity levels can be used to dry the coating formulation onto the microprojections.

For storage and application (in accordance with one embodiment of the invention), the microprojection member 10 is preferably suspended in a retainer ring by adhesive tabs, as described in detail in Co-Pending U.S. application Ser. No. 09/976,762 (Pub. No. 2002/0091357), which is incorporated by reference herein in its entirety.

After placement of the microprojection member 10 in the retainer ring, the microprojection member 10 is applied to the patient's skin. Preferably, the microprojection member 10 is applied to the skin using an impact applicator, such as disclosed in Co-Pending U.S. application Ser. No. 09/976,798, which is incorporated by reference herein in its entirety.

Certain preferred compositions of the invention comprise nucleic acid in the range of approximately 20% to 80% by total dry weight, a stabilizing agent in the range of approximately 20% to 75% by total dry weight, optionally a surface active agent up to approximately 10% by total dry weight, and optionally a buffering agent in the range of approximately 0.5% to 20% by total dry weight. Certain other preferred compositions of the invention comprise nucleic acid in the range of approximately 20% to 80% by total dry weight, a stabilizing agent in the range of approximately 20% to 75% by total dry weight, a DNase inhibitor in the range of approximately 1% to 10% by total dry weight, optionally a surface active agent up to approximately 10% by total dry weight, and optionally a buffering agent in the range of approximately 0.5% to 20% by total dry weight. In certain embodiments of the invention, the compositions and solid coatings do not contain zwitterions with polar residues or derivatives thereof.

The following examples are illustrative of certain embodiments of the invention and should not be considered to limit the scope of the invention.

### EXAMPLE 1

### Evaluation of the Stability of Dry-Coated Plasmid DNA

The stability of plasmid DNA in solution or dry-coated onto glass or titanium and stored at various temperatures was evaluated over time by gel electrophoresis and densitometry. An aqueous stock solution of plasmid DNA (12.15 mg/mL beta-galactosidase expression plasmid having a cytomegalovirus promoter in 2 mM Tris and 1 mM EDTA, pH 7.4) was used for stability evaluation in solution. The same stock solution was dry-coated onto titanium or glass substrates. The coated substrates were dried at room temperature in a vacuum chamber (28 inches mercury gauge) for 2 hours. Each coated substrate was then transferred to a vial, which was caped and stored for various times and temperatures. The dry formulation was then eluted from the substrate in 1 ml TE buffer (10 mM Tris/1 mM EDTA, pH 7.5) by gently shaking for 10 min at room temperature and frozen at −20° C. until analysis. The results are expressed as the percentage of the plasmid DNA that was found to be supercoiled at the time of analysis.

Plasmid DNA dry-coated onto titanium was very unstable when stored at room temperature. Before coating, 97% of the DNA was found supercoiled in the stock solution stored at 4° C. Following coating on a titanium microprojection array (average of 38 pg DNA per 2 cm² array), 62% and 28% of the DNA was found supercoiled, after 1 week and 4 weeks storage at room temperature, respectively.

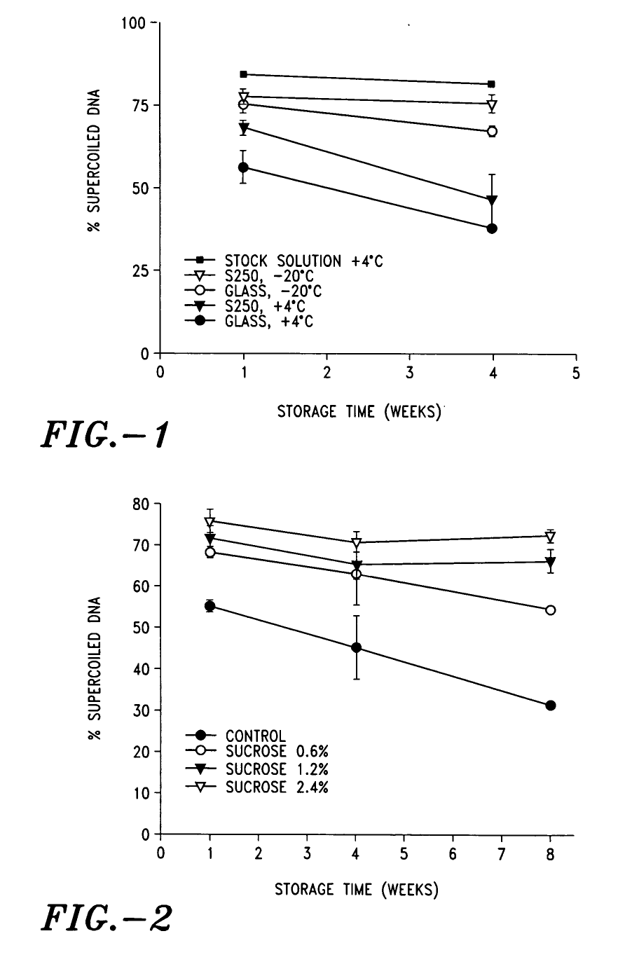

Plasmid DNA dry-coated onto glass or titanium was also unstable when stored at 4° C. The stock solution of plasmid DNA was dry-coated onto either a glass substrate or a titanium microprojection array (S250) and stored at either 4° C. or −20° C. for one to four weeks. A total of about 60 μg DNA was coated per 2 cm² array. As a control, the stock solution was stored at 4° C. for one to four weeks. The percentage of supercoiled plasmid DNA was determined by gel electrophoresis and densitometry after each week. FIG. 1 illustrates that the percentage of supercoiled plasmid DNA dry-coated onto the glass substrate or the titanium microprojection array and stored at 4° C. decreased dramatically over time, indicating that the dry-coated plasmid DNA was quite unstable at 4° C. when coated on a glass or titanium substrate, which contrasts with the relative stability of DNA in the stock solution at this temperature. Thus these results indicate that DNA is unstable in the dry state at 4° C. regardless of the coating substrate. Results also demonstrate that stability is improved from 4° C. to −20° C.

Sucrose improved the stability of dry-coated plasmid DNA stored at 4° C. The stock solution of plasmid DNA, and stock solutions containing 0.6%, 1.2%, or 2.4% sucrose, were dry coated onto titanium discs and stored at 4° C. for 1 to 8 weeks. A total of 60 μg DNA was coated per 2cm² disc. The percentage of supercoiled plasmid DNA was determined each week by gel electrophoresis and densitometry. FIG. 2 illustrates that the percentage of supercoiled plasmid DNA in the control coating containing no sucrose decreased over time. Sucrose reduced the loss of supercoiled plasmid DNA as a function of time in a dose dependant fashion.

The results of the DNA stability studies indicate that the stability of dry-coated plasmid DNA stored at 4° C. is dramatically improved by sucrose in a dose-dependent fashion.

### EXAMPLE 2

### Evaluation of the Ability of Various Substances to Stabilize Plasmid DNA

A number of agents were evaluated for their ability to prevent the loss of supercoiled structure in plasmid DNA dry-coated onto titanium discs. The DNA stock solution used as a control was a 12.5 mg/mL aqueous solution of a plasmid encoding beta galactosidase in 2 mM Tris and 1 mM EDTA, pH 7.4. All other formulations were prepared from the DNA stock solution and contained 10 mg/ml DNA, either with or without 20 mg/ml of a test agent. The following agents were tested: Sucrose (Pfanstiehl, U.S.), Trehalose (Pfanstiehl), D-Mannitol (Sigma, U.S.), Lactose (Pfanstiehl), Dextran with an average mw of 66900 (Sigma), Low molecular weight Hydroxyethylcellulose (HEC, Union carbide, U.S.), Human albumin (Sigma), Glycine (Sigma), NaCl (Sigma), Polyethylene glycol with an average mw of 10000 (PEG 10000, Aldrich, U.S.), Pluronic F127 (Sigma), and glucosaminyl muramyl dipeptide (GMDP, Zao Peptech U.K.)

Titanium discs (2 cm²) were washed in water for 10 min, followed by absolute ethanol for 10 min, followed by acetone for 10 min, and dried at room temperature. 5 ml of each formulation was transferred onto the discs in duplicate. The coated discs were dried at room temperature in a vacuum chamber for 2 hours. Each disc was then transferred to a vial, which was caped and stored at 4° C. for eight weeks. The dry formulation was then eluted from the disc in 1 ml TE buffer (1 mM Tris/1 mM EDTA, pH 7.5) by gently shaking for 10 min at room temperature and frozen at −20° C. until analysis.

Analysis was performed by agarose gel electrophoresis of samples. 200 ng of DNA/lane was run for 105 min at 85V. Integrated Density Values (IDVs) were determined from a gel image that was acquired on an Alphaimager 2200 imaging system. IDV was measured for supercoiled (SC) and non-supercoiled (NSC) bands and the % supercoiled was calculated as (IDV_{SC}/IDVSC+NSC)×100. Results, shown in Table 1, demonstrate that non-reducing sugars (sucrose, trehalose), reducing sugars (lactose), as well as polysaccharides such as dextran, decreased the degradation of dried DNA. Conversely, compounds such as inorganic salts, amino acids, and sugar alcohols did not prevent markedly the degradation of dried DNA.

TABLE I
Effect of various agents on stability of plasmid DNA

% supercoiled

| | Mean | SD |
|---|---|---|
| DNA Solution | 97.2 | |
| Dry DNA | 36.9 | 3.6 |
| Dry DNA + Sucrose | 74.5 | 2.2 |
| Dry DNA + Trehalose | 69.0 | 1.0 |
| Dry DNA + Dextran 66900 | 64.7 | 1.4 |
| Dry DNA + Lactose | 60.4 | 2.7 |
| Dry DNA + PEG 10000 | 48.7 | 11.4 |
| Dry DNA + D-Mannitol | 45.4 | 2.5 |
| Dry DNA + NaCl | 44.3 | 5.7 |
| Dry DNA + GMDP | 32.7 | 0.0 |
| Dry DNA + Pluronic F127 | 29.6 | 0.2 |
| Dry DNA + Human Albumin | 26.2 | 2.4 |
| Dry DNA + HEC | 25.5 | 1.2 |
| Dry DNA + Glycine | 15.7 | 3.7 |

From the foregoing description, one of ordinary skill in the art can easily ascertain that the present invention, among other things, provides an effective and efficient means for enhancing the transdermal flux of a biologically active agent into and through the stratum comeum of a patient.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A solid coating comprising a dried formulation of a stabilizing agent and nucleic acid applied to a solid substrate, wherein said stabilizing agent retards degradation of said dried nucleic acid.

2. The solid coating of claim 1, wherein said solid substrate comprises a microprojection member.

3. The solid coating of claim 2, having a thickness in the range of approximately 1 to 50 micrometers.

4. The solid coating of claim 1, wherein said stabilizing agent is selected from the group consisting of a non-reducing sugar, a polysaccharide, and a reducing sugar.

5. The solid coating of claim 4, wherein said non-reducing sugar is selected from the group consisting of sucrose, trehalose, stachyose, and raffinose.

6. The solid coating of claim 5, wherein said non-reducing sugar comprises sucrose.

7. The solid coating of claim 4, wherein said polysaccharide is selected from the group consisting of dextran, soluble starch, dextrin, and inulin.

8. The solid coating of claim 4, wherein said reducing sugar is selected from the group consisting of apiose, arabinose, lyxose, ribose, xylose, digitoxose, fucose, quercitol, quinovose, rhamnose, allose, altrose, fructose, galactose, glucose, gulose, hamamelose, idose, mannose, tagatose, primeverose, vicianose, rutinose, scillabiose, cellobiose, gentiobiose, lactose, lactulose, maltose, melibiose, sophorose, and turanose.

9. The solid coating of claim 1, wherein said nucleic acid is selected from the group consisting of double-stranded DNA, single-stranded DNA, and RNA.

10. The solid coating of claim 9, wherein said nucleic acid is plasmid DNA.

11. The solid coating of claim 1, wherein said stabilizing agent is in the range of approximately of 10% to 80% by total dry weight of said formulation.

12. The solid coating of claim 1, wherein said nucleic acid is in the range of approximately 20% to 80% by total dry weight of said formulation.

13. The solid coating of claim 1, wherein said formulation further comprises one or more surface active agents up to 10% by total dry weight of said formulation and wherein said one or more surface active agents is selected from the group consisting of polysorbate 20, polysorbate 80, and sodium dodecyl sulfate.

14. The solid coating of claim 1, wherein said formulation further comprises one or more surface active agents up to 10% by total dry weight of said formulation and wherein said one or more surface active agents is selected from the group consisting of negatively charged surfactants, positively charged surfactants and neutral surfactants.

15. The solid coating of claim 14, wherein said negatively charged surfactant comprises cetylpyridinium chloride.

16. The solid coating of claim 14, wherein said positively charged surfactant is selected from the group consisting of cetylpyridinium chloride, TMAC and benzalkonium chloride.

17. The solid coating of claim 14, wherein said neutral surfactant is selected from the group consisting of polysorbate, sorbitan and laureth.

18. The solid coating of claim 13, wherein said formulation further comprises a buffering agent in the range of approximately 0.5% to 20% by total dry weight and wherein said buffering agent is selected from the group consisting of phosphoric acid, citric acid, and TRIS.

19. The solid coating of claim 1, wherein said formulation further comprises a buffering agent in the range of approximately 0.5% to 20% by total dry weight and wherein said buffering agent is selected from the group consisting of phosphoric acid, citric acid, and TRIS.

20. The solid coating of claim 1, wherein said formulation comprises said nucleic acid in the range of approximately 20% to 80% by total dry weight and said stabilizing agent in the range of approximately 10% to 80% by total dry weight.

21. The solid coating of claim 2, wherein said nucleic acid comprises DNA and further comprising a DNase inhibitor, wherein said DNase inhibitor retards degradation of the DNA following delivery of said solid coating into or through skin using said microprojection member.

22. The solid coating of claim 21, wherein said DNase inhibitor is selected from the group consisting of aurintricarboxylic acid, EDTA, EGTA, propamidine, and DMI-2.

23. The solid coating of claim 21, wherein said DNase inhibitor is in the range of approximately 1% to 20% by total dry weight of said formulation.

24. The solid coating of claim 21, wherein said formulation further comprises up to 10% by total dry weight of one or more surface active agents and wherein said one or more surface active agents is selected from the group consisting of polysorbate 20, polysorbate 80, and sodium dodecyl sulfate.

25. The solid coating of claim 24, wherein said formulation further comprises a buffering agent in the range of approximately 0.5% to 20% by total dry weight and wherein said buffering agent is selected from the group consisting of phosphoric acid, citric acid, and TRIS.

26. The solid coating of claim 21, wherein said formulation further comprises a buffering agent in the range of approximately 0.5% to 20% by total dry weight and wherein said buffering agent is selected from the group consisting of phosphoric acid, citric acid, and TRIS.

27. The solid coating of claim 21, wherein said formulation comprises said nucleic acid in the range of approximately 20% to 80% by total dry weight, said stabilizing agent in the range of approximately 10% to 80% by total dry weight and said Dnase inhibitor in the range of approximately 1% to 20% by total dry weight.

28. The solid coating of claim 1, wherein said formulation includes a vasoconstrictor.

29. The solid coating of claim 28, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

30. The solid coating of claim 1, wherein said formulation includes a pathway patency modulator.

31. The solid coating of claim 30, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

32. The solid coating of claim 1, wherein said formulation includes an antioxidant.

33. The solid coating of claim 32, wherein said antioxidant is selected from the group consisting of sodium citrate, citric acid, ethylene-dinitrilo-tetraacetic acid (EDTA), ascorbic acid, methionine, and sodium ascorbate.

34. A method for retarding the degradation of a nucleic acid comprising the steps of mixing a formulation of said nucleic acid with a stabilizing agent and dry-coating said formulation onto a solid substrate, wherein said stabilizing agent retards degradation of said nucleic acid.

35. The method of claim 34, wherein the step of dry-coating said formulation onto a solid substrate comprises coating a microprojection member.

36. The method of claim 35, further comprising the step of applying said microprojection member to a subject to transdermally deliver said nucleic acid.

37. The method of claim 34, wherein the step of dry-coating said formulation onto a solid substrate comprises coating said solid substrate to a thickness in the range of approximately 1 to 50 microns.

38. The method of claim 34, wherein the step of mixing a formulation of said nucleic acid with a stabilizing agent comprises mixing a stabilizing agent selected from the group consisting of a non-reducing sugar, a polysaccharide, and a reducing sugar.

39. The method of claim 34, wherein the step of mixing a formulation of said nucleic acid with a stabilizing agent comprises mixing a nucleic acid selected from the group consisting of double-stranded DNA, single-stranded DNA, and RNA.

40. The method of claim 34, further comprising the step of adding to said formulation up to 10% by total dry weight of one or more surface active agents selected from the group consisting of polysorbate 20, polysorbate 80, and sodium dodecyl sulfate.

41. The method of claim 34, further comprising the step of adding to said formulation a buffering agent in the range of approximately 0.5% to 20% by total dry weight, wherein said buffering agent is selected from the group consisting of phosphoric acid, citric acid, and TRIS.

42. The method of claim 34, wherein said nucleic acid comprises DNA, further comprising the step of adding to said formulation a DNase inhibitor.

43. The method of claim 42, wherein the step of adding said DNase inhibitor comprises adding a DNase inhibitor selected from the group consisting of aurintricarboxylic acid, EDTA, EGTA, propamidine, and DMI-2.

44. The method of claim 43, wherein the step of adding said DNase inhibitor comprises adding said DNase inhibitor in the range of approximately 1% to 20% by total dry weight.

45. The method of claim 42, wherein the step of dry-coating said formulation onto a solid substrate comprises coating a microprojection member.

46. The method of claim 45, further comprising the step of applying said microprojection member to a subject to transdermally deliver said nucleic acid, wherein said DNase inhibitor retards degradation of the nucleic acid following delivery.
